(19) United States
(12) Patent Application Publication
(10) Pub. No.: US 2010/0330130 A1
(43) Pub. Date: Dec. 30, 2010

(54) SUBSTANTIALLY PURE IMATINIB OR A PHARMACEUTICALLY ACCEPTABLE SALT THEREOF

(75) Inventors: Mayur Devjibhai KHUNT, Gujarat (IN); Nilesh Sudhir PATIL, Maharashtra (IN); Haushabhau Shivaji PAGIRE, Maharashtra (IN); Nitin Sharadchandra PRADHAN, Maharashtra (IN)

Correspondence Address: CANTOR COLBURN LLP, 20 Church Street, 22nd Floor, Hartford, CT, 06103, US

(73) Assignee: ACTAVIS GROUP PTC EHF, Hafnarfjordur (IS)

(21) Appl. No.: 12/782,234

(22) Filed: May 18, 2010

(30) Foreign Application Priority Data
May 22, 2009 (IN) 1182/CHE/2009

(51) Int. Cl. A61K 31/496; C07D 403/14; A61K 9/00; A61P 35/00

(52) U.S. Cl. 424/400; 514/252.18; 544/295

(57) ABSTRACT

Provided herein are impurities of imatinib, N-(2-Methyl-5-methylamino-phenyl)-N-(4-pyridin-3-yl-pyrimidin-2-yl)-formamide (formamide impurity) and 4-[4-(Imidazole-1-carbonyl)-piperazin-1-ylmethyl]-N-[4-methyl-3-(4-pyridin-3-yl-pyrimidin-2-ylamino)-phenyl]-benzamide (carbonylimidazole impurity), and processes for the preparation and isolation thereof. Provided further herein is a highly pure imatinib or a pharmaceutically acceptable salt thereof substantially free of formamide and carbonylimidazole impurities, processes for the preparation thereof, and pharmaceutical compositions comprising highly pure imatinib or a pharmaceutically acceptable salt thereof substantially free of impurities. Disclosed also herein is a process for preparing substantially pure α-form of imatinib mesylate.

### CROSS REFERENCE TO RELATED APPLICATION

This application claims the benefit of priority to Indian provisional application No. 1182/CHE/2009, filed on May 22, 2009, which is incorporated herein by reference in its entirety.

### FIELD OF THE DISCLOSURE

Disclosed herein are impurities of imatinib or a pharmaceutically acceptable salt thereof, and processes for the preparation and isolation thereof. Disclosed further herein is a highly pure imatinib or a pharmaceutically acceptable salt thereof substantially free of impurities, processes for the preparation thereof, and pharmaceutical compositions comprising highly pure imatinib or a pharmaceutically acceptable salt thereof substantially free of impurities. Disclosed also herein is a process for preparing substantially pure α-form of imatinib mesylate.

### BACKGROUND

U.S. Pat. No. 5,521,184 discloses a variety of N-phenyl-2-pyrimidine-amine derivatives, processes for their preparation, pharmaceutical compositions comprising the derivatives, and method of use thereof. These compounds are useful in the treatment of tumoral diseases. Among them, imatinib, 4-[(4-methyl-1-piperazinyl)methyl]-N-[4-methyl-3-[[4-(3-pyridinyl)-2-pyrimidinyl]amino]phenyl]benzamide, is a protein-tyrosine kinase inhibitor that is especially useful in the treatment of various types of cancers and for the treatment of atherosclerosis, thrombosis, restenosis, or fibrosis. Imatinib can also be used for the treatment of non-maligant diseases. Imatinib is usually administered orally in the form of a suitable salt, e.g., in the form of imatinib mesylate. Imatinib is sold by Novartis as Gleevec™ capsules containing imatinib mesylate equivalent to 100 mg of imatinib free base. Imatinib is represented by the following structural formula I:

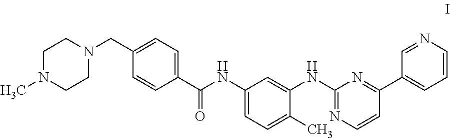

Various processes for the preparation of imatinib and related compounds are disclosed in U.S. Pat. No. 5,521,184; U.S. Patent Application Nos. 2006/0149061, 2007/0197545, 2008/0207904; and PCT Publication Nos. WO 2003/066613 A1, WO 2004/074502 A2, WO 2004/108699 A1, WO 2006/061332 A1 and WO 2006/071130 A2.

As per the process described in the U.S. Pat. No. 5,521,184 (hereinafter referred to as the '184 patent), imatinib is prepared by the reaction of 2-methyl-5-nitroaniline with an aqueous solution of cyanamide in the presence of nitric acid in ethanol, to produce 2-methyl-5-nitrophenyl guanidine nitrate. This product is reacted with 3-dimethylamino-1-(3-pyridinyl)-2-propen-1-one in the presence of sodium hydroxide in isopropanol to produce N-(2-methyl-5-nitrophenyl)-4-(3-pyridinyl)-2-pyrimidineamine, followed by reduction using hydrogen in the presence of Pd/C catalyst in ethyl acetate to produce N-(5-amino-2-methylphenyl)-4-(3-pyridinyl)-2-pyrimidineamine, which is then condensed with 4-(4-methyl-piperazinomethyl)benzoyl chloride in pyridine. The crude product obtained is then subjected to column chromatographic purifications using a solvent system containing chloroform and methanol to yield imatinib.

Imatinib obtained by the process described in the '184 patent does not have satisfactory purity for pharmaceutical use. Unacceptable amounts of impurities are generally formed along with imatinib. The yield of imatinib obtained is very poor and the process involves column chromatographic purifications. Methods involving column chromatographic purifications are generally undesirable for large-scale operations, thereby making the process commercially unfeasible. Moreover, the '184 patent involves the use of highly hazardous materials like pyridine as a solvent for the condensation of N-(5-amino-2-methylphenyl)-4-(3-pyridinyl)-2-pyrimidineamine with 4-(4-methyl-piperazinomethyl)benzoyl chloride. Use of a solvent such as pyridine in the final stage of a synthetic process is not advisable for scale up operations, since it is very difficult to remove residual traces thereof from the final product.

PCT Publication No. WO 2003/066613 A1 (hereinafter referred to as the '613 application) describes several synthetic routes for preparing imatinib. According to one synthetic process, imatinib is prepared by the reaction of 4-(4-methyl-piperazin-1-ylmethyl)-benzoic acid methyl ester with 3-nitro-4-methyl-aniline to give N-(4-methyl-3-nitrophenyl)-4-(4-methyl-piperazin-1-ylmethyl)-benzamide, which is subsequently reduced to obtain N-(3-amino-4-methyl-phenyl)-4-(4-methyl-piperazin-1-ylmethyl)-benzamide. This product is reacted with cyanamide in a mixture of concentrated hydrochloric acid solution and n-butanol to produce N-(3-guanidino-4-methyl-phenyl)-4-(4-methyl-piperazin-1-ylmethyl)-benzamide, which is then reacted with 3-dimethylamino-1-pyridin-3-yl-propenone to yield imatinib.

According to another synthetic process as described in the '613 application, imatinib is prepared by the reaction of 3-bromo-4-methyl-aniline with 4-(4-methyl-piperazin-1-ylmethyl)-benzoic acid methyl ester to afford N-(3-bromo-4-methyl-phenyl)-4-(4-methyl-piperazin-1-ylmethyl)-benzamide, which is reacted with 4-(3-pyridyl)-2-pyrimidine amine to yield imatinib.

PCT Publication No. WO 2004/074502 A2 (hereinafter referred to as the '502 application) describes a process for the preparation of imatinib by the reaction of N-(2-methyl-5-aminophenyl-4-(3-pyridyl)-2-pyrimidineamine with 4-(4-methyl-piperazinomethyl)benzoyl chloride dihydrochloride in dimethylformamide to produce imatinib trihydrochloride monohydrate, which is then treated with aqueous ammonia to produce imatinib.

The process described in the '502 application also suffers from drawbacks since imatinib obtained by the process does not have satisfactory purity, unacceptable amounts of impurities are formed along with imatinib, and the yield of imatinib is poor.

It is known that synthetic compounds can contain extraneous compounds or impurities resulting from their synthesis or degradation. The impurities can be unreacted starting materials, by-products of the reaction, products of side reactions, or degradation products. Generally, impurities in an active pharmaceutical ingredient (API) may arise from degradation of the API itself, or during the preparation of the API. Impurities in imatinib or any active pharmaceutical ingredient (API) are undesirable and might be harmful.

Regulatory authorities worldwide require that drug manufacturers isolate, identify and characterize the impurities in their products. Furthermore, it is required to control the levels of these impurities in the final drug compound obtained by the manufacturing process and to ensure that the impurity is present in the lowest possible levels, even if structural determination is not possible.

The product mixture of a chemical reaction is rarely a single compound with sufficient purity to comply with pharmaceutical standards. Side products and byproducts of the reaction and adjunct reagents used in the reaction will, in most cases, also be present in the product mixture. At certain stages during processing of the active pharmaceutical ingredient, the product is analyzed for purity, typically, by HPLC, TLC or GC analysis, to determine if it is suitable for continued processing and, ultimately, for use in a pharmaceutical product. Purity standards are set with the intention of ensuring that an API is as free of impurities as possible, and, thus, are as safe as possible for clinical use. The United States Food and Drug Administration guidelines recommend that the amounts of some impurities are limited to less than 0.1 percent.

Generally, impurities are identified spectroscopically and by other physical methods, and then the impurities are associated with a peak position in a chromatogram (or a spot on a TLC plate). Thereafter, the impurity can be identified by its position in the chromatogram, which is conventionally measured in minutes between injection of the sample on the column and elution of the particular component through the detector, known as the “retention time” (“Rt”). This time period varies daily based upon the condition of the instrumentation and many other factors. To mitigate the effect that such variations have upon accurate identification of an impurity, practitioners use “relative retention time” (“RRT”) to identify impurities. The RRT of an impurity is its retention time divided by the retention time of a reference marker.

It is known by those skilled in the art, the management of process impurities is greatly enhanced by understanding their chemical structures and synthetic pathways, and by identifying the parameters that influence the amount of impurities in the final product.

There is a need for highly pure imatinib or a pharmaceutically acceptable salt thereof substantially free of impurities, as well as processes for preparing thereof.

Imatinib mesylate can exist in different polymorphic forms, which differ from each other in terms of stability, physical properties, spectral data and methods of preparation.

Various polymorphic forms, including hydrated and solvated forms, of imatinib mesylate designated Forms α, β, H1, α2, δ, ε, I, II, F, G, H, I, K, IV, V, VI, VII, VIII, IX, X, XI, XIII, XIV, XV, XVI and amorphous forms are apparently disclosed in U.S. Pat. No. 6,894,051 B1, U.S. Pat. No. 7,300,938 B2, PCT Publication Nos. WO 2005/077933, WO 2005/095379, WO 2006/054314, WO 2006/024863, WO 2006/048890, WO 2007/023182, and WO 2007/136510.

U.S. Pat. No. 6,894,051 B1 (hereinafter referred to as the '051 patent) discloses two crystalline modifications (α-form and β-form) of imatinib mesylate, processes for their preparation, and characterizes the modifications by powder X-ray diffraction (P-XRD), differential scanning calorimetry (DSC) and crystal morphology.

Polymorphism is defined as the ability of a substance to exist as two or more crystalline phases that have different arrangement and/or conformations of the molecule in the crystal lattice. Different polymorphs may differ in their physical properties such as melting point, solubility, X-ray diffraction patterns, and the like. Although these differences disappear once the compound is dissolved, they can appreciably influence the pharmaceutically relevant properties of the solid form, such as handling properties, dissolution rate and stability. Such properties can significantly influence the processing, shelf life, and commercial acceptance of a polymorph. It is therefore important to investigate all solid forms of a pharmaceutical compound, including all polymorphic forms, and to determine the stability, dissolution and flow properties of each polymorphic form.

Solvent medium and mode of isolation play very important roles in obtaining a polymorphic form over another.

Still, there is a strong technical and commercial desire to develop a modified process for the preparation of substantially pure α-form of imatinib mesylate.

### SUMMARY

In one aspect, provided herein is an isolated formamide compound, N-(2-methyl-5-methylamino-phenyl)-N-(4-pyridin-3-yl-pyrimidin-2-yl)-formamide, having the following structural formula 1:

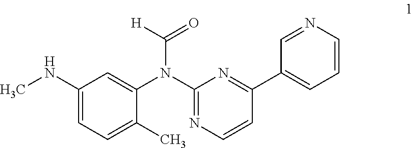

or an acid addition salt thereof.

In another aspect, provided herein is an impurity of imatinib, formamide impurity, N-(2-methyl-5-methylamino-phenyl)-N-(4-pyridin-3-yl-pyrimidin-2-yl)-formamide, of formula 1.

In another aspect, encompassed herein is a process for synthesizing and isolating the formamide compound of formula 1, also referred to as the “formamide impurity”.

In another aspect, provided herein is an isolated carbonylimidazole compound, 4-[4-(imidazole-1-carbonyl)-piperazin-1-ylmethyl]-N-[4-methyl-3-(4-pyridin-3-yl-pyrimidin-2-ylamino)-phenyl]-benzamide, having the following structural formula 2:

or

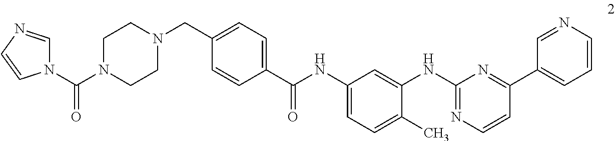

an acid addition salt thereof.

In another aspect, provided herein is an impurity of imatinib, carbonylimidazole impurity, 4-[4-(imidazole-1-carbonyl)-piperazin-1-ylmethyl]-N-[4-methyl-3-(4-pyridin-3-yl-pyrimidin-2-ylamino)-phenyl]-benzamide, of formula 2.

In another aspect, encompassed herein is a process for synthesizing and isolating the carbonylimidazole compound of formula 2, also referred to as the “carbonylimidazole impurity”.

In another aspect, provided herein is a highly pure imatinib or a pharmaceutically acceptable salt thereof substantially free of formamide and carbonylimidazole impurities.

In yet another aspect, encompassed herein is a process for preparing the highly pure imatinib or a pharmaceutically acceptable salt thereof substantially free of formamide and carbonylimidazole impurities.

Preferable pharmaceutically acceptable salts of Imatinib include, but are not limited to, hydrochloride, hydrobromide, oxalate, maleate, fumarate, mesylate, besylate, tosylate, tartrate, and more preferably mesylate.

In another aspect, provided herein is a pharmaceutical composition comprising highly pure imatinib or a pharmaceutically acceptable salt thereof substantially free of formamide and carbonylimidazole impurities, and one or more pharmaceutically acceptable excipients.

In still another aspect, provided herein is a pharmaceutical composition comprising highly pure imatinib or a pharmaceutically acceptable salt thereof substantially free of formamide and carbonylimidazole impurities made by the process disclosed herein, and one or more pharmaceutically acceptable excipients.

In still further aspect, encompassed is a process for preparing a pharmaceutical formulation comprising combining highly pure imatinib or a pharmaceutically acceptable salt thereof substantially free of formamide and carbonylimidazole impurities with one or more pharmaceutically acceptable excipients.

In another aspect, the highly pure imatinib or a pharmaceutically acceptable salt thereof substantially free of formamide and carbonylimidazole impurities disclosed herein for use in the pharmaceutical compositions has a D₉₀ particle size of less than or equal to about 500 microns, specifically about 1 micron to about 300 microns, and most specifically about 10 microns to about 150 microns.

In another aspect, encompassed herein is a process for preparing α-form of imatinib mesylate with high purity.

### BRIEF DESCRIPTION OF THE DRAWINGS

FIG. 1 is a powder X-ray diffraction (XRD) pattern of α-form of imatinib mesylate prepared according to the process disclosed in Example 3 (Sample A).

FIG. 2 is a powder X-ray diffraction (XRD) pattern of α-form of imatinib mesylate prepared according to the process disclosed in Example 3 (Sample B).

FIG. 3 is a powder X-ray diffraction (XRD) pattern of α-form of imatinib mesylate prepared according to the process disclosed in Example 4.

FIG. 4 is a powder X-ray diffraction (XRD) pattern of α-form of imatinib mesylate prepared according to the process disclosed in Example 5.

FIG. 5 is a powder X-ray diffraction (XRD) pattern of α-form of imatinib mesylate prepared according to the process disclosed in Example 6.

FIG. 6 is a powder X-ray diffraction (XRD) pattern of α-form of imatinib mesylate prepared according to the process disclosed in Example 7.

### DETAILED DESCRIPTION

According to one aspect, there is provided a formamide compound, N-(2-methyl-5-methylamino-phenyl)-N-(4-pyridin-3-yl-pyrimidin-2-yl)-formamide, having the following structural formula 1:

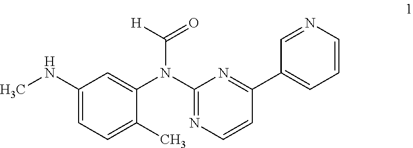

or an acid addition salt thereof.

According to another aspect, there is provided an impurity of imatinib, formamide impurity, N-(2-methyl-5-methylamino-phenyl)-N-(4-pyridin-3-yl-pyrimidin-2-yl)-formamide, of formula 1.

The formamide impurity has been identified, isolated and synthesized. The formamide impurity was detected and resolved from imatinib by HPLC with an RRt of 0.72. The structure of the compound of formula 1 was deduced with the aid of ¹H, ¹³C NMR and IR spectroscopy and FAB mass spectrometry. The parent ion at 319 is consistent with the assigned structure.

The formamide compound (formula 1) disclosed herein is characterized by data selected from a ¹H NMR (300 MHz, CDCl₃) δ (ppm): 2.17 (s, 3H, N-Methyl), 2.32 (s, 3H, Methyl of aniline), 7.01 (s, 1H, Ar—H of aniline), 7.14 (s, 1H, Ar—H of aniline), 7.16 (d, 1H, pyrimidine), 7.18-7.20 (d, 1H, pyridine), 7.42 (s, 1H, Ar—H of aniline), 7.46 (s, 1H, Ar—H of pyrimidine), 8.38 (s, 1H, NH of N-Methyl), 8.43-8.51 (dd, 2H, Ar—H of pyridine), 8.72 (s, 1H, pyridine), 9.27 (s, 1H, N-Formyl); MS: EI⁺ m/z (MH+): 319; and IR spectra on KBr having absorption bands at about 1672 cm⁻¹ (N-Formyl).

The acid addition salts of formamide compound can be derived from a therapeutically acceptable acid such as hydrochloric acid, hydrobromic acid, sulfuric acid, nitric acid, phosphoric acid, acetic acid, propionic acid, oxalic acid, succinic acid, maleic acid, fumaric acid, methanesulfonic acid, benzenesulfonic acid, toluenesulfonic acid, citric acid, and tartaric acid.

Preferable pharmaceutically acceptable acid addition salts of a formamide compound include, but are not limited to, hydrochloride, hydrobromide, oxalate, sulphate, phosphate, fumarate, succinate, maleate, mesylate, besylate, tosylate and tartrate.

According to another aspect, there is provided an isolated formamide impurity. The formamide impurity formed during the synthesis of imatinib or a pharmaceutically acceptable salt thereof can be isolated by subjecting the imatinib or a pharmaceutically acceptable salt thereof that contains the formamide impurity to column chromatography. The column chromatography comprises using a silica gel, as a stationary phase, and a gradient of eluents that remove the formamide impurity from the column on which it adsorbed.

The present inventors have found that the formamide compound of formula 1 is formed as an impurity, in an amount of about 4% to about 6% as measured by HPLC, during the synthesis of imatinib free base.

In one embodiment, the formamide compound of formula 1 is prepared as per the process exemplified in Example 8 as disclosed herein.

According to another aspect, there is provided a carbonylimidazole compound, 4-[4-(imidazole-1-carbonyl)-piperazin-1-ylmethyl]-N-[4-methyl-3-(4-pyridin-3-yl-pyrimidin-2-ylamino)-phenyl]-benzamide, having the following structural formula 2:

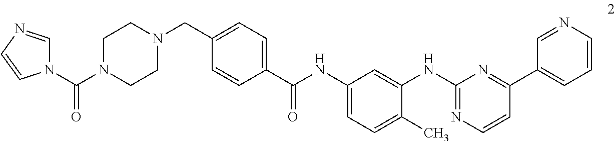

or an acid addition salt thereof.

According to another aspect, there is provided an impurity of imatinib, carbonylimidazole impurity, 4-[4-(imidazole-1-carbonyl)-piperazin-1-ylmethyl]-N-[4-methyl-3-(4-pyridin-3-yl-pyrimidin-2-ylamino)-phenyl]-benzamide, of formula 2.

The carbonylimidazole impurity has been identified, isolated and synthesized. The carbonylimidazole impurity was detected and resolved from imatinib by HPLC with an RRt of 0.96. The structure of the compound of formula 2 was deduced with the aid of ¹H, ¹³C NMR and IR spectroscopy and FAB mass spectrometry. The parent ion M⁺ at 573 is consistent with the assigned structure.

The carbonylimidazole compound (formula 2) disclosed herein is characterized by data selected from a ¹H NMR (300 MHz, CDCl₃) δ (ppm): 2.34 (s, 3H, methyl of aniline), 2.49-2.53 (t, 4H, piperazine); 3.59 (t, 4H, piperazine), 3.63 (s, 2H, —CH₂ of benzamide), 7.08 (s, 1H, NH), 7.10 (d, 1H, Ar—H of aniline), 7.15-7.19 (dd, 2H, pyridine), 7.21 (d, 1H, pyrimidine), 7.30-7.34 (dd, 1H, Ar—H of aniline), 7.38 (s, 1H, Ar—H of aniline), 7.40-7.43 (s, 2H, Ar—H of benzamide), 7.84 (s, 1H, NH of amide), 7.86-7.87 (s, 2H, Ar—H of benzamide), 8.20 (s, 1H, Ar—H of aniline), 8.45 (d, 1H, pyridine), 8.46-8.50 (dd, 1H, pyridine), 8.58 (d, 1H, pyridine), 8.67 (dd, 1H, pyridine); MS: EI⁺ m/z (MH⁺): 573; and IR spectra on KBr having absorption bands at about 1700 cm⁻¹ (Ketone of imidazole), 1660 cm⁻¹ (Ketone of benzamide).

The acid addition salts of carbonylimidazole compound can be derived from a therapeutically acceptable acid selected from the group as described above.

According to another aspect, there is provided an isolated carbonylimidazole impurity. The carbonylimidazole impurity formed during the synthesis of imatinib or a pharmaceutically acceptable salt thereof can be isolated by subjecting the imatinib or a pharmaceutically acceptable salt thereof that contains the carbonylimidazole impurity to column chromatography. The column chromatography comprises using a silica gel, as a stationary phase, and a gradient of eluents that remove carbonylimidazole impurity from the column on which it adsorbed.

The present inventors have found that the carbonylimidazole compound of formula 2 is formed as an impurity, in an amount of about 0.16% to about 0.5% as measured by HPLC, during the synthesis of imatinib free base.

In one embodiment, the carbonylimidazole compound of formula 2 is prepared as per the process exemplified in Example 9 as disclosed herein.

Regarding the specific RRt values of impurities disclosed herein, it is well known to a person skilled in the art that the RRt values may vary from sample to sample due to, inter alia, instrument errors (both instrument to instrument variation and the calibration of an individual instrument) and differences in sample preparation. Thus, it has been generally accepted by those skilled in the art that independent measurement of an identical RRt value can differ by amounts of up to ±0.02.

Thus there is a need for a method for determining the level of impurities in imatinib samples and removing the impurities.

Extensive research and experimentation was carried out by the present inventors to reduce the level of the formamide and carbonylimidazole impurities in imatinib. As a result, it has been found that the formation of formamide and carbonylimidazole impurities in the synthesis of imatinib or a pharmaceutically acceptable salt thereof can be controlled or substantially removed by contacting crude imatinib base with sodium dihydrogen phosphate in a suitable solvent under appropriate reaction conditions while adjusting pH to 4-5, and isolating highly pure imatinib free base substantially free of formamide and carbonylimidazole impurities, and then converting the pure imatinib free base obtained into a pharmaceutically acceptable salt thereof. Moreover, it has been surprisingly observed that the use of acids such as hydrochloric acid, sulfuric acid, etc., in the same pH range (4-5) could not effectively remove these impurities.

According to another aspect, there is provided a highly pure imatinib or a pharmaceutically acceptable salt thereof substantially free of formamide and carbonylimidazole impurities.

As used herein, “highly pure imatinib or a pharmaceutically acceptable salt thereof substantially free of formamide and carbonylimidazole impurities” refers to imatinib or a pharmaceutically acceptable salt thereof comprising the formamide impurity in an amount of less than about 1.5 area-% and the carbonylimidazole impurity in an amount of less than about 0.15 area-% as measured by HPLC. Specifically, the imatinib, as disclosed herein, contains less than about 1.2 area-% of the formamide impurity and less than about 0.1 area-% of the carbonylimidazole impurity, more specifically less than about 0.9 area-% of the formamide impurity and less than about 0.05 area-% of the carbonylimidazole impurity, still more specifically less than about 0.05 area-% of the formamide impurity and less than about 0.02 area-% of the carbonylimidazole impurity, and most specifically is essentially free of the formamide and carbonylimidazole impurities.

In one embodiment, the highly pure imatinib or a pharmaceutically acceptable salt thereof disclosed herein comprises a formamide impurity in an amount of about 0.01 area-% to about 1.5 area-% and a carbonylimidazole impurity in an amount of about 0.01 area-% to about 0.15 area-%, and specifically comprises the formamide impurity in an amount of about 0.01 area-% to about 1.0 area-% and the carbonylimidazole impurity in an amount of about 0.01 area-% to about 0.1 area-%, as measured by HPLC.

In another embodiment, the highly pure imatinib or a pharmaceutically acceptable salt thereof disclosed herein has a total purity of greater than about 98%, specifically greater than about 99%, more specifically greater than about 99.9%, and most specifically greater than about 99.95% as measured by HPLC. For example, the purity of the imatinib or a pharmaceutically acceptable salt thereof is about 98% to about 99.9%, or about 99.5% to about 99.99%.

In another embodiment, the highly pure imatinib or a pharmaceutically acceptable salt thereof disclosed herein is essentially free of one, or both, of the formamide and carbonylimidazole impurities.

The term “imatinib or a pharmaceutically acceptable salt thereof essentially free of one, or both, of the formamide and carbonylimidazole impurities” refers to imatinib or a pharmaceutically acceptable salt thereof contains a non-detectable amount of one, or both, of the formamide and carbonylimidazole impurities as measured by HPLC.

Exemplary pharmaceutically acceptable salts of imatinib include, but are not limited to, hydrochloride, hydrobromide, oxalate, maleate, fumarate, mesylate, besylate, tosylate and tartrate. A specific pharmaceutically acceptable salt of imatinib is imatinib mesylate.

According to another aspect, there is provided a purification process for obtaining highly pure imatinib or a pharmaceutically acceptable salt thereof substantially free of the formamide and carbonylimidazole impurities, comprising:

a) contacting crude imatinib base with a dihydrogen phosphate in a solvent medium comprising water and a first organic solvent to produce a biphasic reaction mixture;

b) separating the aqueous layer from the biphasic reaction mixture and adding a second organic solvent to the separated aqueous layer to produce a reaction mixture;

c) combining the reaction mixture obtained in step-(b) with a base to produce a reaction mass; and

d) isolating and/or recovering the highly pure imatinib free base substantially free of formamide and carbonylimidazole impurities as a solid from the reaction mass;

e) optionally, suspending the highly pure imatinib free base obtained in step-(d) in a third organic solvent and recovering the imatinib free base from the suspension;

f) optionally, converting the highly pure imatinib obtained in step-(d) or step-(e) into a pharmaceutically acceptable salt thereof.

In one embodiment, the process disclosed herein or any one of the process steps can be repeated any number of times until the substantial removal of the formamide and carbonylimidazole impurities and to provide the desired purity.

Exemplary first organic solvents used in step-(a) include, but are not limited to, an alcohol, a ketone, a chlorinated hydrocarbon, a hydrocarbon, an ester, a nitrile, an ether, a polar aprotic solvent, and mixtures thereof. The term solvent also includes mixtures of solvents.

In one embodiment, the first organic solvent is selected from the group consisting of methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, methylene chloride, ethylene dichloride, chloroform, n-pentane, n-hexane, n-heptane, cyclohexane, toluene, xylene, tetrahydrofuran, dioxane, diethyl ether, diisopropyl ether, monoglyme, diglyme, N,N-dimethylformamide, N,N-dimethylacetamide, dimethylsulfoxide, and mixtures thereof.

Specifically, the first organic solvent is selected from the group consisting of methanol, ethanol, isopropyl alcohol, acetone, methylene chloride, N,N-dimethylformamide, N,N-dimethylacetamide, dimethylsulfoxide, and mixtures thereof; and more specifically methanol, methylene chloride, dimethylsulfoxide, and mixtures thereof.

Exemplary dihydrogen phosphates used in step-(a) include, but are not limited to, sodium dihydrogen phosphate, potassium dihydrogen phosphate, and ammonium dihydrogen phosphate. A specific dihydrogen phosphate is sodium dihydrogen phosphate.

The dihydrogen phosphate in step-(a) may be used directly or in the form of an aqueous solution.

In one embodiment, the contacting in step-(a) is carried out under stirring at a temperature of below about reflux temperature of the solvent medium used for at least 10 minutes, specifically at a temperature of about 0° C. to about 80° C. for about 15 minutes to about 15 hours, and more specifically at about 20° C. to about 60° C. for about 20 minutes to about 5 hours. In another embodiment, the pH of the reaction mass is adjusted between 4 and 5 during the addition of dihydrogen phosphate.

In one embodiment, the crude imatinib base used in step-(a) may be in the form of a solid, a solution, a suspension or a reaction mass containing crude imatinib base.

In another embodiment, the solution of crude imatinib base is prepared by dissolving crude imatinib base in the first organic solvent or in the solvent medium comprising water and the first organic solvent, or obtaining an existing solution from a previous processing step.

In one embodiment, the crude imatinib free base is dissolved in the first organic solvent or in the solvent medium comprising water and the first organic solvent at a temperature below about reflux temperature of the solvent or solvent medium used, specifically at about 15° C. to about 110° C., and more specifically at about 20° C. to about 80° C.

In another embodiment, the suspension of crude imatinib base is prepared by suspending crude imatinib base in the first organic solvent or in the solvent medium comprising water and the first organic solvent while stirring at a temperature below boiling temperature of the solvent or the solvent medium used. In one embodiment, the suspension is stirred at a temperature of about 15° C. to about 110° C. for at least 30 minutes, and more specifically at about 25° C. to about 80° C. from about 1 hour to about 10 hours.

In another embodiment, the solution or the suspension or the reaction mass containing crude imatinib base is prepared by reacting 4-(4-methyl-piperazinomethyl)benzoic acid dihydrochloride with N-(5-Amino-2-methylphenyl)-4-(3-pyridinyl)-2-pyrimidineamine in the presence of N,N′-carbonyldiimidazole, in a reaction inert solvent, preferably dimethylsulfoxide, under suitable conditions to produce a reaction mass containing crude imatinib base, and optionally followed by usual work up such as washings, extractions, evaporations, etc. In one embodiment, the work-up includes dissolving, suspending or extracting the resulting crude imatinib base in the first organic solvent or in the solvent medium comprising water and the first organic solvent at a temperature below boiling temperature of the solvent or the solvent medium used, specifically at about 15° C. to about 110° C., and more specifically at about 20° C. to about 80° C.

Alternatively, the solution or suspension of crude imatinib base is prepared by treating a crude imatinib acid addition salt with a base to liberate crude imatinib base followed by extracting, dissolving or suspending the imatinib base in the first organic solvent or in the solvent medium comprising water and the first organic solvent at a temperature below boiling temperature of the solvent or the solvent medium used, specifically at about 15° C. to about 110° C., and more specifically at about 20° C. to about 80° C.

In another embodiment, the acid addition salt of imatinib is derived from a therapeutically acceptable acid such as hydrochloric acid, hydrobromic acid, sulfuric acid, nitric acid, phosphoric acid, acetic acid, propionic acid, oxalic acid, succinic acid, maleic acid, fumaric acid, methanesulfonic acid, benzenesulfonic acid, toluenesulfonic acid, citric acid, and tartaric acid.

The treatment of an acid addition salt with a base is carried out in a solvent and the selection of solvent is not critical. A wide variety of solvents such as chlorinated solvents, alcohols, ketones, hydrocarbon solvents, esters, ether solvents etc., can be used.

The base used herein is an inorganic base or an organic base. In one embodiment, the base is used in the form of an aqueous solution. Specific organic bases are triethyl amine, dimethyl amine and tert-butyl amine.

In another embodiment, the base is an inorganic base. Exemplary inorganic bases include, but are not limited to, aqueous ammonia; hydroxides, alkoxides, carbonates and bicarbonates of alkali or alkaline earth metals. Specific inorganic bases are aqueous ammonia, sodium hydroxide, calcium hydroxide, magnesium hydroxide, potassium hydroxide, lithium hydroxide, sodium carbonate, potassium carbonate, lithium carbonate, sodium bicarbonate, potassium bicarbonate, sodium tert-butoxide, sodium isopropoxide and potassium tert-butoxide, and more specifically aqueous ammonia, sodium hydroxide, potassium hydroxide, sodium carbonate and potassium carbonate.

The aqueous layer obtained in step-(b) is optionally washed with the first organic solvent prior to the addition of the second organic solvent.

Exemplary second organic solvents used in step-(b) include, but are not limited to, an alcohol, a ketone, a chlorinated hydrocarbon, a hydrocarbon, an ester, a nitrile, an ether, a polar aprotic solvent, and mixtures thereof. The term solvent also includes mixtures of solvents.

In one embodiment, the second organic solvent is selected from the group consisting of methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, methylene chloride, ethylene dichloride, chloroform, n-pentane, n-hexane, n-heptane, cyclohexane, toluene, xylene, tetrahydrofuran, dioxane, diethyl ether, diisopropyl ether, monoglyme, diglyme, N,N-dimethylformamide, N,N-dimethylacetamide, dimethylsulfoxide, and mixtures thereof.

Specifically, the second organic solvent is an ester solvent selected from the group consisting of ethyl acetate, methyl acetate, isopropyl acetate, tert-butyl methyl acetate, ethyl formate, and mixtures thereof; and more specifically ethyl acetate.

The addition of the second organic solvent in step-(b) is, for example, carried out drop wise or in one portion or in more than one portion. In one embodiment, the addition is carried out at a temperature of below boiling temperature of the solvent used, specifically at about 15° C. to about 110° C., and more specifically at about 20° C. to about 80° C.

The base used in step-(c) is an organic or an inorganic base selected from the group as described above. Specific inorganic bases are aqueous ammonia, sodium hydroxide, potassium hydroxide, sodium carbonate and potassium carbonate; and more specifically aqueous ammonia.

Combining of the reaction mixture with base in step-(c) is done in a suitable order, for example, the reaction mixture is added to the base, or alternatively, the base is added to the reaction mixture. The addition is, for example, carried out drop wise or in one portion or in more than one portion. The addition is specifically carried out at a temperature of below about 60° C., and more specifically at a temperature of about 20° C. to about 55° C. After completion of addition process, the resulting mass is stirred at a temperature of about 20° C. to about 70° C. for about 10 minutes to about 15 hours, and more specifically at about 25° C. to about 55° C. for about 30 minutes to about 5 hours.

In one embodiment, the pH of the reaction mixture is adjusted between 8 and 10 during the addition of base.

The isolation of highly pure imatinib free base in step-(d) is carried out, for example, by forcible or spontaneous crystallization.

Spontaneous crystallization refers to crystallization without the help of an external aid, such as seeding, cooling etc., and forcible crystallization refers to crystallization with the help of an external aid.

Forcible crystallization is initiated by methods such as cooling, seeding, partial removal of the solvent from the solution, by combining an anti-solvent with the solution, or a combination thereof.

In one embodiment, the crystallization is carried out by cooling the solution at a temperature of below 20° C. for at least 15 minutes, specifically at about 0° C. to about 20° C. for about 30 minutes to about 20 hours, and more specifically at about 0° C. to about 15° C. for about 2 hours to about 10 hours.

The recovery of highly pure imatinib free base in step-(d) is accomplished by techniques such as filtration, filtration under vacuum, decantation, centrifugation, or a combination thereof. In one embodiment, the imatinib free base is recovered by filtration employing a filtration media of, for example, a silica gel or celite.

In another embodiment, the solid imatinib free base obtained in step-(d) is optionally washed with solvents such as water, alcohols, and mixtures thereof prior to suspending in the third organic solvent.

Exemplary third organic solvents used in step-(e) include, but are not limited to, an aliphatic or aromatic hydrocarbon, an ether, an alcohol, a ketone, and mixtures thereof. The term solvent also includes mixtures of solvents.

In one embodiment, the third organic solvent is selected from the group consisting of n-pentane, n-hexane, n-heptane, cyclohexane, toluene, xylene, tetrahydrofuran, dioxane, diethyl ether, diisopropyl ether, monoglyme, diglyme, methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, and mixtures thereof.

Specifically, the third organic solvent is a hydrocarbon solvent selected from the group consisting of n-pentane, n-hexane, n-heptane, cyclohexane, toluene, xylene, and mixtures thereof; and more specifically n-heptane.

In another embodiment, the suspension obtained in step-(e) is stirred at a temperature below about reflux temperature of the solvent for at least about 10 minutes, specifically at about 20° C. to about 110° C. for about 30 minutes to about 20 hours, and specifically at about 25° C. to about 80° C. for about 1 hour to about 5 hours.

The recovery of highly pure imatinib free base in step-(e) is accomplished by techniques as described above.

Pharmaceutically acceptable salts of imatinib in step-(f) can be prepared in high purity by using the highly pure imatinib substantially free of impurities obtained by the method disclosed herein, by known methods, or by the methods disclosed hereinafter.

Specific pharmaceutically acceptable salts of imatinib include, but are not limited to, hydrochloride, hydrobromide, oxalate, maleate, fumarate, mesylate, besylate, tosylate, and tartrate. A most specific pharmaceutically acceptable salt of imatinib is imatinib mesylate and still more specifically in the form of crystalline form-α.

The highly pure imatinib or a pharmaceutically acceptable salt thereof obtained by the above process may be further dried in, for example, a Vacuum Tray Dryer, a Rotocon Vacuum Dryer, a Vacuum Paddle Dryer or a pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, the drying is carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer, and the like. Drying equipment selection is well within the ordinary skill in the art.

According to another aspect, there is provided a process preparing crystalline form-α of imatinib mesylate, comprising:

a) providing a solution of imatinib mesylate in a solvent selected from the group consisting of N,N-dimethylformamide, N,N-dimethylacetamide, and mixtures thereof;

b) optionally, filtering the solution to remove insoluble matter;

c) precipitating crystalline form-α of imatinib mesylate by combining the solution obtained in step-(a) or step-(b) with an anti-solvent selected from the group consisting of isopropyl alcohol, acetone, and mixtures thereof; and

d) optionally, seeding the solution in step-(c) with crystalline form-α prior to or after the addition of anti-solvent;

e) recovering the crystalline form-α of imatinib mesylate in substantially pure form.

The term “substantially pure α-form of imatinib mesylate” refers to the α-form of imatinib mesylate having purity greater than about 99%, specifically greater than about 99.5%, more specifically greater than about 99.8% and still more specifically greater than about 99.9% (measured by HPLC).

The crystalline form-α of imatinib mesylate obtained by the process disclosed herein is stable, consistently reproducible and has good flow properties, and is particularly suitable for bulk preparation and handling, and hence, the α-form of imatinib mesylate obtained by the process disclosed herein is suitable for formulating imatinib mesylate.

The imatinib mesylate crystalline form-α obtained by the process disclosed herein is substantially free from other crystalline forms, particularly crystalline form-β.

In one embodiment, the imatinib mesylate crystalline form-α obtained by the process disclosed herein specifically is essentially free of crystalline form-ft “Essentially free of crystalline form-β of imatinib mesylate” means that no crystalline form-β can be detected in the imatinib mesylate crystalline form-α within the limits of a powder X-ray diffractometer.

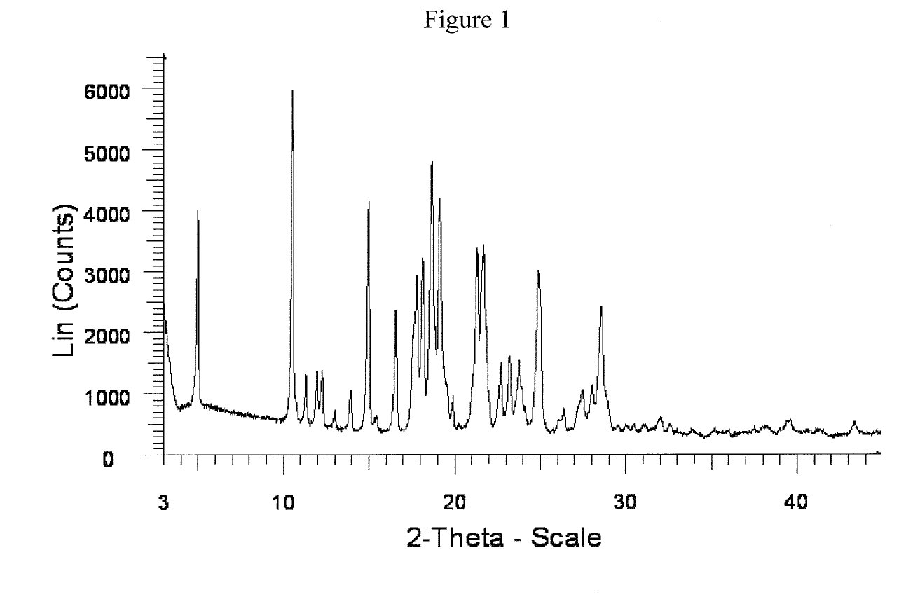

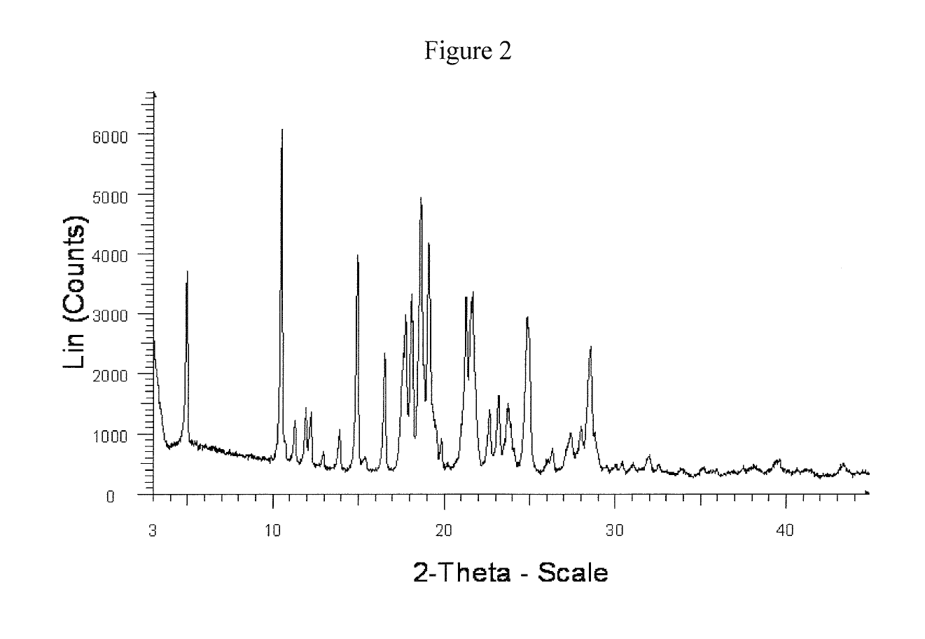

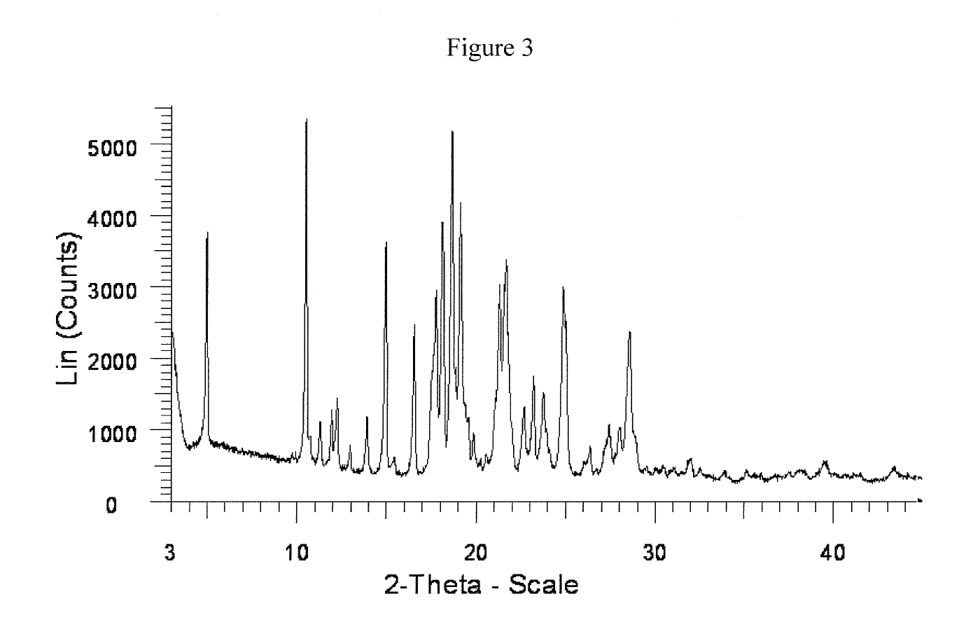

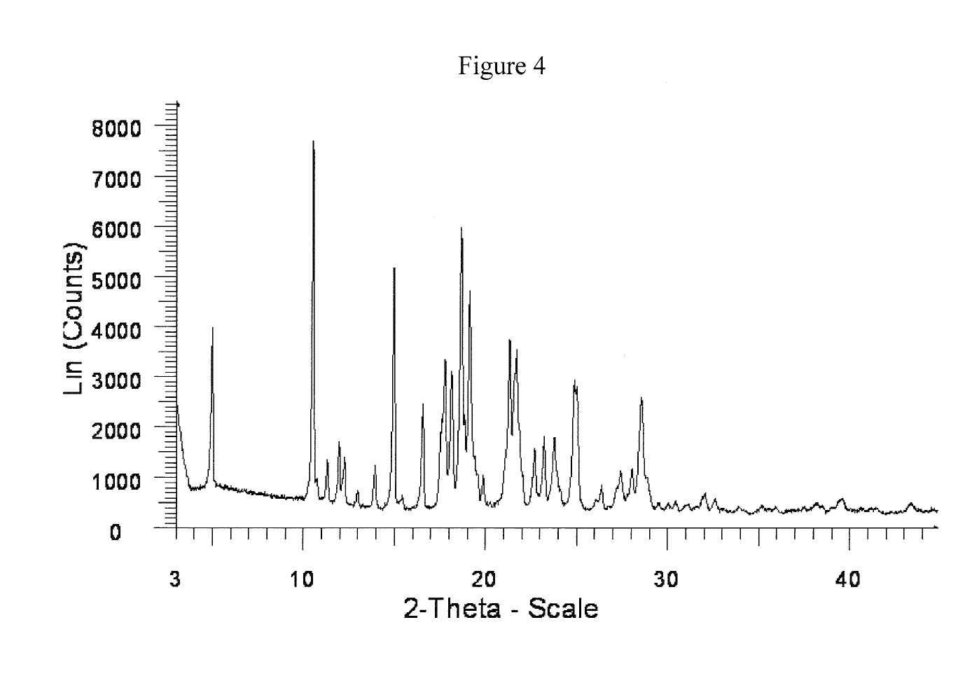

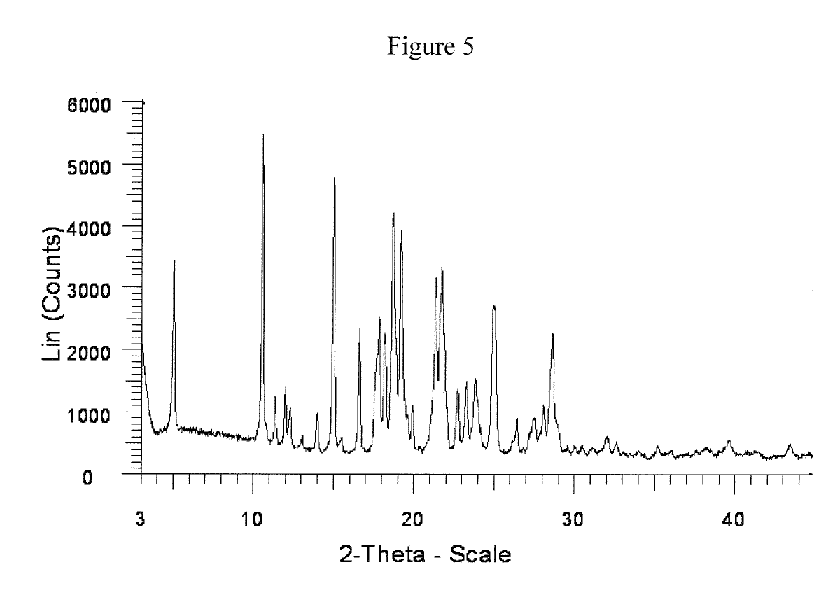

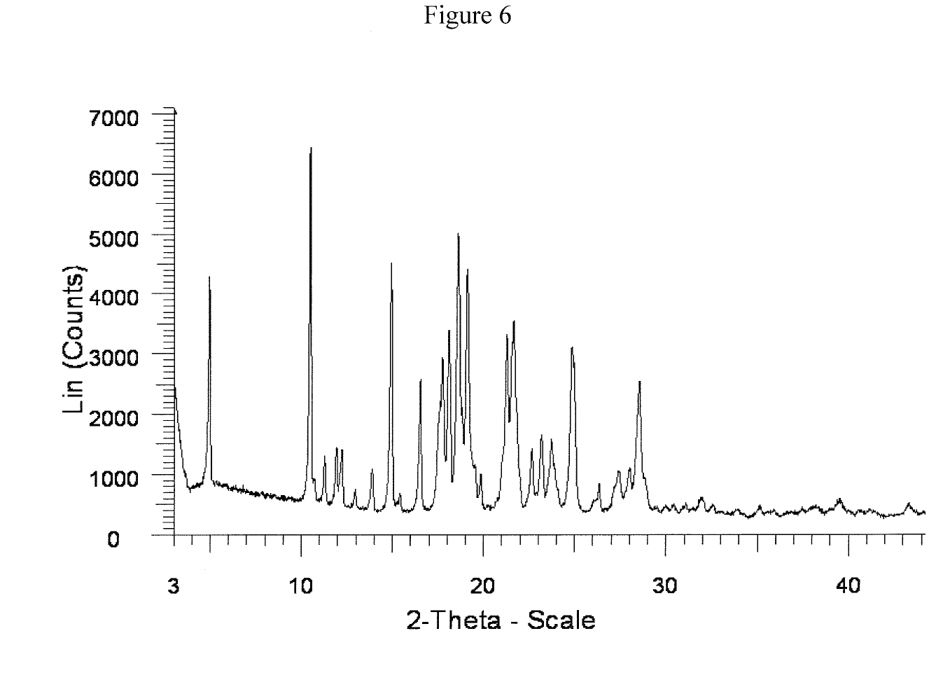

In another embodiment, the imatinib mesylate crystalline form-α obtained by the process disclosed herein is characterized by a powder X-ray diffraction pattern substantially in accordance with any one of the FIGS. 1 to 6.

Step-(a) of providing a solution of imatinib mesylate includes dissolving imatinib mesylate in the solvent, or obtaining an existing solution from a previous processing step.

In one embodiment, the imatinib mesylate is dissolved in the solvent at a temperature of below about reflux temperature of the solvent used, specifically at about 40° C. to about 80° C., and still more specifically at about 50° C. to about 70° C.

In another embodiment, the solution in step-(a) is prepared by admixing imatinib base, methanesulfonic acid and the solvent to obtain a mixture; and stirring the mixture to obtain a solution of imatinib mesylate. In yet another embodiment, the mixture is stirred at a temperature of below about reflux temperature of the solvent used for at least 15 minutes, specifically at about 40° C. to about 80° C. for about 20 minutes to about 10 hours, and still more specifically at about 50° C. to about 70° C. for about 30 minutes to about 5 hours.

The solution obtained in step-(a) is optionally subjected to carbon treatment or silica gel treatment. The carbon treatment or silica gel treatment is carried out by methods known in the art, for example, by stirring the solution with finely powdered carbon or silica gel at a temperature of below about 70° C. for at least 15 minutes, specifically at a temperature of about 40° C. to about 70° C. for at least 30 minutes; and filtering the resulting mixture through hyflo to obtain a filtrate containing imatinib mesylate by removing charcoal or silica gel. Specifically, the finely powdered carbon is an active carbon. A specific mesh size of silica gel is 40-500 mesh, and more specifically 60-120 mesh.

The solution obtained in step-(a) is optionally stirred at a temperature of about 30° C. to the reflux temperature of the solvent used for at least 20 minutes, and specifically at a temperature of about 40° C. to the reflux temperature of the solvent used from about 30 minutes to about 4 hours.

In one embodiment, the amount of solvent employed in step-(a) is about 3 volumes to about 6 volumes, and specifically about 4 volumes to about 5 volumes with respect to the quantity of imatinib free base.

Combining of the solution with anti-solvent in step-(c) is done in a suitable order, for example, the solution is added to the anti-solvent, or alternatively, the anti-solvent is added to the solution. The addition is, for example, carried out drop wise or in one portion or in more than one portion. The addition is specifically carried out at a temperature below about reflux temperature of the solvent used, and more specifically at a temperature of about 40° C. to about 80° C. After completion of addition process, the resulting mass is stirred at a temperature of about 20° C. to about 110° C. for about 10 minutes to about 15 hours, and more specifically at about 40° C. to about 90° C. for about 30 minutes to about 5 hours.

In another embodiment, the amount of anti-solvent employed in step-(c) is about 6 volumes to about 35 volumes, and specifically about 8 volumes to about 32 volumes with respect to the quantity of imatinib free base.

The recovery of crystalline form-α of imatinib mesylate in step-(e) is accomplished by the techniques as described above.

The highly pure imatinib mesylate crystalline form-α obtained by the above process may be further dried as per the methods described hereinabove.

Further encompassed herein is the use of the highly pure imatinib or a pharmaceutically acceptable salt thereof substantially free of formamide and carbonylimidazole impurities for the manufacture of a pharmaceutical composition together with a pharmaceutically acceptable carrier.

A specific pharmaceutical composition of highly pure imatinib or a pharmaceutically acceptable salt thereof substantially free of formamide and carbonylimidazole impurities is selected from a solid dosage form and an oral suspension.

In one embodiment, the highly pure imatinib or a pharmaceutically acceptable salt thereof substantially free of formamide and carbonylimidazole impurities has a D₉₀ particle size of less than or equal to about 500 microns, specifically about 1 micron to about 300 microns, and most specifically about 10 microns to about 150 microns.

In another embodiment, the particle sizes of the highly pure imatinib or a pharmaceutically acceptable salt thereof substantially free of formamide and carbonylimidazole impurities are produced by a mechanical process of reducing the size of particles which includes any one or more of cutting, chipping, crushing, milling, grinding, micronizing, trituration or other particle size reduction methods known in the art, to bring the solid state form to the desired particle size range.

According to another aspect, there is provided a method for treating a patient suffering from tumoral diseases, comprising administering a therapeutically effective amount of the highly pure imatinib or a pharmaceutically acceptable salt thereof substantially free of formamide and carbonylimidazole impurities, or a pharmaceutical composition that comprises a therapeutically effective amount of highly pure imatinib or a pharmaceutically acceptable salt thereof substantially free of formamide and carbonylimidazole impurities, along with pharmaceutically acceptable excipients.

According to another aspect, there is provided pharmaceutical compositions comprising highly pure imatinib or a pharmaceutically acceptable salt thereof substantially free of formamide and carbonylimidazole impurities prepared according to the process disclosed herein and one or more pharmaceutically acceptable excipients.

According to another aspect, there is provided a process for preparing a pharmaceutical formulation comprising combining highly pure imatinib or a pharmaceutically acceptable salt thereof substantially free of formamide and carbonylimidazole impurities prepared according to the process disclosed herein, with one or more pharmaceutically acceptable excipients.

Yet in another embodiment, pharmaceutical compositions comprise at least a therapeutically effective amount of highly pure imatinib or a pharmaceutically acceptable salt thereof substantially free of formamide and carbonylimidazole impurities. Such pharmaceutical compositions may be administered to a mammalian patient in a dosage form, e.g., solid, liquid, powder, elixir, aerosol, syrups, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, syrup, troches, sachets, suspensions, powders, lozenges, elixirs and the like. The highly pure imatinib or a pharmaceutically acceptable salt thereof substantially free of formamide and carbonylimidazole impurities may also be administered as suppositories, ophthalmic ointments and suspensions, and parenteral suspensions, which are administered by other routes.

The pharmaceutical compositions further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described herein.

In one embodiment, capsule dosage forms contain highly pure imatinib or a pharmaceutically acceptable salt thereof substantially free of formamide and carbonylimidazole impurities within a capsule which may be coated with gelatin. Tablets and powders may also be coated with an enteric coating. Suitable enteric coating agents include phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, the coating agents may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions described herein may contain diluents such as cellulose-derived materials such as powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols such as mannitol and sorbitol, acrylate polymers and copolymers, as well as pectin, dextrin and gelatin.

Other excipients include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

### Instrumental Details:

### X-Ray Powder Diffraction (P-XRD):

The X-Ray powder diffraction was measured by an X-ray powder Diffractometer equipped with CuKα-radiations (40 kV, 40 mA) in wide-angle X-ray Diffractometer of BRUKER axs, D8 ADVANCE. The sample was analyzed using the following instrument parameters: measuring range=3-45° 2-theta; step width=0.01579°; and measuring time per step=0.11 sec.

### High Performance Liquid Chromatography (HPLC):

The HPLC purity was measured by high performance liquid chromatography by using Shimadzu LC 2010 AHT HPLC system having UV detector with LC solution chromatography software or its equivalent under the following conditions:

Column: XBridge C18, 250×4.6 mm×5.0μ), Make: Waters,
Part No: 186003117

### Detector: UV at 230 nm

Injection volume: 10.0 μL
Run time: 40 min
Column temperature: 40° C.
Flow rate: 1.0 ml/min
Diluent: Mobile phase B

### Buffer Preparation:

Di sodium hydrogen phosphate (1.4 g) was taken in 1000 ml of water and pH was adjusted to 8.00 with diluted H₃PO₄, followed by filtration through a 0.22 μm or finer porosity membrane and degassing.

Mobile Phase-A: Buffer (100%)

Mobile Phase-B: Buffer:Methanol (30:70).

### Gradient Program:

| Time (min) | (%) Mobile phase-A | (%) Mobile phase-B |
|---|---|---|
| 0 | 50 | 50 |
| 3 | 50 | 50 |
| 15 | 10 | 90 |
| 30 | 10 | 90 |
| 32 | 50 | 50 |
| 40 | 50 | 50 |

The following examples are given for the purpose of illustrating the present invention and should not be considered as limitation on the scope or spirit of the invention.

### Reference Example

### Preparation of Crude Imatinib Base

A mixture of 4-(4-methyl-piperazinomethyl)benzoic acid dihydrochloride (277.1 g) and dimethylsulfoxide (880 g) was stirred, followed by the addition of N,N′-carbonyldiimidazole (147 g) under a nitrogen atmosphere. The temperature of the resulting mixture was raised to 40° C., and then the mixture was stirred for 2 hours at 40° C. To the resulting mass was added portion wise N-(5-amino-2-methylphenyl)-4-(3-pyridinyl)-2-pyrimidineamine (200 g) under stirring, the temperature of the reaction was raised to 45-50° C. and then stirred for 2 to 3 hours. Upon completion of the reaction, the reaction mass was cooled to 30-35° C., followed by quenching the mass with a mixture of water (8000 ml) and ethyl acetate (720 ml) under stirring at a temperature of 30-35° C., and the stirring was continued for 30 minutes. Aqueous ammonia solution (250 ml) was added drop-wise to the quenched reaction mixture at 30-35° C. while adjusting the pH between 8 and 9, and the resulting mass was stirred for 2 hours. The separated solid was filtered, washed with water (1000 ml) and suction dried for 2 hours. The resulting wet material was followed by the addition of n-heptane (1400 ml) and then stirred for 1 to 2 hours at ambient temperature. The resulting solid was filtered, washed with n-heptane (100 ml) and suction dried for 30 minutes and then dried the material in vacuum oven for 8 to 12 hours at 70-75° C. to give 289 g of crude imatinib base (Purity by HPLC: 92.69%).

### Content of Impurities Measured by HPLC:

1. N-(2-Methyl-5-methylamino-phenyl)-N-(4-pyridin-3-yl-pyrimidin-2-yl)-formamide (formamide impurity) at 0.72 RRT: 4.06%.

2. 4-[4-(Imidazole-1-carbonyl)-piperazin-1-ylmethyl]-N-[4-methyl-3-(4-pyridin-3-yl-pyrimidin-2-ylamino)-phenyl]-benzamide (carbonylimidazole impurity) at 0.96 RRT: 0.17%.

3. Amine intermediate, N-(5-Amino-2-methylphenyl)-4-(3-pyridinyl)-2-pyrimidine amine, at 0.69 RRT: 2.19%.

### EXAMPLES

### Example 1

### Step-I: Purification of Imatinib Base

Dichloromethane (500 ml) and methanol (10 ml) were added to crude imatinib base (50 g, Purity by HPLC: 92.69%; Formamide impurity: 4.06%; Carbonylimidazole impurity: 0.17%; Amine intermediate: 2.19%) at 20-25° C. and stirred for 20 minutes. To the resulting suspension was added water (3000 ml) and 20% aqueous sodium dihydrogen phosphate solution (600 ml) followed by stirring for 10 minutes. The resulting layers were separated and the aqueous layer was washed with dichloromethane (250 ml). The resulting aqueous layer was separated, followed by the addition of ethyl acetate (150 ml) and aqueous ammonia (150 ml) and the resulting mass was stirred for 2 hours at 20-25° C. The separated solid was filtered, washed with water (500 ml) and then dried the solid in air oven at 60-65° C. for 12 hours to yield 31 g of pure imatinib base (Purity by HPLC: 98.61%).

### Content of Impurities Measured by HPLC:

1. Formamide impurity at 0.72 RRT: 1.02%.

2. Carbonylimidazole impurity at 0.96 RRT: Not detected.

3. Amine intermediate at 0.69 RRT: 0.08%.

### Step-II: Preparation of pure Imatinib mesylate (α-Form)

Imatinib base (50 g, obtained in step-I) was suspended in N,N-dimethylacetamide (150 ml) at 25-30° C. to form a suspension. A mixture of methanesulfonic acid (9.7 g) and N,N-dimethylacetamide (50 ml) was added to the above suspension at 25-30° C. followed by heating the reaction mixture at 60-65° C. to provide a clear solution. The resulting clear solution was filtered through a hyflow bed and the hyflow bed was washed with dimethylacetamide (25 ml). The resulting clear filtrate was heated at 50-55° C., followed by the addition of isopropyl alcohol (1575 ml). The reaction mixture was further heated at 80-85° C., followed by the addition of seed of imatinib mesylate α-Form (0.3 g) and the resulting mixture was maintained for 4 hours at 80-85° C. A small portion of the reaction mass was collected at this stage, and the solid was filtered and washed with isopropyl alcohol (10 ml). The resulting sample (sample 1) was analyzed by HPLC. The remaining portion of the reaction mass was gradually cooled to 20-25° C. Another small portion of the reaction mass was collected at this stage, and the solid was filtered and washed with isopropyl alcohol (10 ml). The resulting sample (sample 2) was analyzed by HPLC. The remaining portion of the reaction mass was stirred for 12 hours at 20-25° C., filtered the solid and washed with isopropyl alcohol (200 ml), followed by drying the solid at 85-90° C. for 10 to 12 hours to give 40 g of pure imatinib mesylate (sample 3).

### HPLC Analysis:

Sample 1: Formamide impurity=0.08%; Carbonylimidazole impurity=0.01%; Purity of compound=99.77%.

Sample 2: Formamide impurity=0.11%; Carbonylimidazole impurity=Not detected; Purity of compound=99.69%.

Sample 3: Formamide impurity=0.11%; Carbonylimidazole impurity=Not detected; Purity of compound=99.60%.

### Example 2

### Preparation of Pure Imatinib Base

A mixture of 4-(4-methyl-piperazinomethyl)benzoic acid dihydrochloride (8.65 g) and dimethylsulfoxide (25 g) was stirred and followed by the addition of N,N′-carbonyldiimidazole (4.53 g) under nitrogen atmosphere. The temperature of the resulting mixture was raised to 40° C. and then stirred for 2 hours at 40° C. To the resulting mass was added portion wise N-(5-Amino-2-methylphenyl)-4-(3-pyridinyl)-2-pyrimidineamine (6.25 g) under stirring, the temperature of the reaction was raised to 45-50° C. and then stirred for 5 hours. Upon completion of the reaction, the reaction mass was cooled to 30-35° C., followed by quenching the mass with water (276 ml) and sodium dihydrogen phosphate (50 g) under stirring at a temperature of 30-35° C., and the stirring was continued for 20 minutes. The resulting mass was washed two times with dichloromethane (2×100 ml) and the aqueous layer was separated. Ethyl acetate (22 ml) and aqueous ammonia (15 ml) were added to the aqueous layer and then stirred for 2 hours. The separated solid was filtered, washed with water (100 ml) and then the solid was dried in a vacuum oven for 12 hours at 70-75° C. to yield 8.5 g of pure imatinib base (Purity by HPLC: 98.90%).

### Content of Impurities Measured by HPLC:

1. Formamide impurity at 0.72 RRT: 0.87%.

2. Carbonylimidazole impurity at 0.96 RRT: 0.01%.

3. Amine intermediate at 0.69 RRT: Not Detected.

### Example 3

### Preparation of α-Form of Imatinib Mesylate

Imatinib base (3 g) was suspended in N,N-dimethylacetamide (10 ml), followed by the addition of a mixture of methanesulfonic acid (0.58 g) and N,N-dimethylacetamide (5 ml) at 20-25° C. The resulting mixture was heated at 55-60° C. to form a clear solution, followed by the addition of isopropyl alcohol (30 ml), and then stirred for 3 hours at 55-60° C. A small portion of the reaction mass was collected at this stage, and the solid was filtered and washed with isopropyl alcohol (2.5 ml), followed by drying in air oven at 75-80° C. The resulting sample (sample A, Dry weight: 1.5 g) was analyzed by powder X-ray diffraction (P-XRD). The remaining portion of the reaction mass cooled to 20-25° C. and stirred for 12 hours at 20-25° C. The separated solid was filtered and washed with isopropyl alcohol (2.5 ml), followed by drying in air oven at 75-80° C. to give imatinib mesylate α-Form (sample B, Dry weight: 1.2 g).

### Analytical Result:

Both the samples of imatinib mesylate (sample A & B) exist in crystalline Form-αand confirmed by powder X-ray diffraction patterns substantially in accordance with FIGS. 1 & 2. The imatinib mesylate crystalline form-α obtained by this process dose not contain crystalline form-β within the limits of a powder X-ray diffractometer.

### Example 4

### Preparation of α-Form of Imatinib Mesylate

N,N-Dimethylacetamide (10 ml) and methanesulfonic acid (0.38 g) were added to imatinib base (2 g) at 20-25° C. and the mixture was heated at 55-60° C. for a time sufficient to form a clear solution. Acetone (20 ml) was added to the above solution, followed by the addition of seed (0.2 g, imatinib mesylate crystalline form-α) and then stirred for 1 hour at 50° C. The separated solid was filtered, washed with acetone (5 ml) and then dried the solid in air oven at 75 to 80° C. to give 1.9 g of imatinib mesylate crystalline form-α.

### Example 5

### Preparation of α-Form of Imatinib Mesylate

N,N-Dimethylformamide (10 ml) and methanesulfonic acid (0.38 g) were added to imatinib base (2 g) at 20-25° C. and the mixture was heated at 60-65° C. until to form a clear solution. Isopropyl alcohol (20 ml) was added to the above solution, followed by the addition of seed (0.2 g, imatinib mesylate crystalline form-α) and then stirred for 1 hour at 45-50° C. The separated solid was filtered, washed with isopropyl alcohol (5 ml) and then dried the solid in air oven at 75 to 80° C. to give 2.1 g of imatinib mesylate crystalline form-α.

### Example 6

### Preparation of α-Form of Imatinib Mesylate

Imatinib base (4 g) was suspended in N,N-dimethylacetamide (16 ml), followed by the addition of methanesulfonic acid (0.77 g) and N,N-dimethylacetamide (4 ml) at 20-25° C. The resulting mixture was heated at 55-60° C. to form a clear solution. Isopropyl alcohol (40 ml) was added to the above solution, followed by the addition of seed (0.2 g, imatinib mesylate crystalline form-α) and then stirred for 3 hours at 55-60° C. The separated solid was filtered, washed with isopropyl alcohol (5 ml) and then dried the solid in air oven at 75-80° C. to give 4.2 g of imatinib mesylate crystalline form-α.

### Example 7

### Preparation of α-Form of Imatinib Mesylate

Imatinib base (10 g) was suspended in N,N-dimethylacetamide (45 ml), followed by the addition of methanesulfonic acid (1.9 g) and N,N-dimethylacetamide (5 ml) at 20-25° C. The resulting mixture was heated at 55-60° C. to form a clear solution. Isopropyl alcohol (100 ml) was added to the above solution, followed by the addition of seed (0.2 g, imatinib mesylate crystalline form-α), and then stirred for 3 hours at 55-60° C. The separated solid was filtered, washed with isopropyl alcohol (10 ml) and then dried the solid in air oven at 75-80° C. to give 9.8 g of imatinib mesylate crystalline form-α.

### Example 8

### Preparation of N-(2-Methyl-5-methylamino-phenyl)-N-(4-pyridin-3-yl-pyrimidin-2-yl)-formamide (Formamide compound or Formamide impurity)

A mixture of 4-(4-methyl-piperazinomethyl)benzoic acid dihydrochloride (277.1 g), dimethylsulfoxide (880 g) and carbonyldiimidazole (147 g) was stirred for 30 minutes at 25-30° C. The resulting mixture was heated at 40-45° C. and followed by the addition of N-(5-amino-2-methylphenyl)-4-(3-pyridinyl)-2-pyrimidineamine (200 g). The reaction mixture was further heated at 50-55° C. and maintained for 2 hours. The resulting mass was cooled to 20-25° C. and followed by quenching with a mixture of water (8000 ml), ethyl acetate (720 ml), and aqueous ammonia (250 ml). The resulting slurry was stirred for 2 hours at 25-30° C. for 2 hours. The separated solid was filtered, washed with water (500 ml) and then dried the solid in air oven at 65-70° C. for 10 to 12 hours. The dried material was subjected to column chromatography to isolate the desired impurity (Yield: 6.6 g, Purity by HPLC: 99.35%).

### Example 9

### Preparation of 4-[4-(Imidazole-1-carbonyl)-piperazin-1-ylmethyl]-N-[4-methyl-3-(4-pyridin-3-yl-pyrimidin-2-ylamino)-phenyl]-benzamide (Carbonylimidazole compound or Carbonylimidazole impurity)

N,N-Dimethylsulfoxide (60 ml) and N,N-carbonyldiimidazole (6.08 g) were added to N-[4-methyl-3-(4-pyridin-3-yl-pyrimidin-2-ylamino)-phenyl]-4-(piperazin-1-ylmethyl)-benzamide (15 g) and the resulting mixture was stirred for 6 hours at 25-30° C. Water (500 ml) was added to the resulting mass and followed by extracting with dichloromethane (200 ml×2). The organic solvent was distilled out from the resulting mass under vacuum, followed by the addition of ethyl acetate (100 ml) and heating at reflux for 2 hours. The solid obtained was filtered and washed with ethyl acetate (50 ml) and then dried the solid in air oven at 65° C. for 8 to 10 hours to produce 17.5 g of 4-[4-(imidazole-1-carbonyl)-piperazin-1-ylmethyl]-N-[4-methyl-3-(4-pyridin-3-yl-pyrimidin-2-ylamino)-phenyl]-benzamide (Purity by HPLC: 97.05%).

Unless otherwise indicated, the following definitions are set forth to illustrate and define the meaning and scope of the various terms used to describe the invention herein.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia, alginic acid, tragacanth, carboxymethylcellulose sodium, polyvinylpyrrolidone, compressible sugar (e.g., NuTab), ethylcellulose, gelatin, liquid glucose, methylcellulose, pregelatinized starch, starch, polyethylene glycol, guar gum, polysaccharide, bentonites, sugars, invert sugars, poloxamers (PLURONIC™ F68, PLURONIC™ F127), collagen, albumin, celluloses in non-aqueous solvents, polypropylene glycol, polyoxyethylene-polypropylene copolymer, polyethylene ester, polyethylene sorbitan ester, polyethylene oxide, microcrystalline cellulose, combinations thereof and other material known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, microcrystalline cellulose (e.g., Avicel™), carsium (e.g., Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxylpropylcellulose, hydroxypropylmethylcellulose phthalate, noncrystalline cellulose, magnesium aluminum silicate, triethanolamine, polyvinyl alcohol, and polyvinylpyrrolidone (PVP).

The term “crude imatinib or a pharmaceutically acceptable salt thereof” as used herein refers to imatinib or a pharmaceutically acceptable salt thereof containing formamide impurity in an amount of greater than about 1.5 area-% and/or carbonylimidazole impurity in an amount of greater than about 0.15 area-% as measured by HPLC.

As used herein, the term, “detectable” refers to a measurable quantity measured using an HPLC method having a detection limit of 0.01 area-%.

As used herein, in connection with amount of impurities in imatinib or a pharmaceutically acceptable salt thereof, the term “not detectable” means not detected by the herein described HPLC method having a detection limit for impurities of 0.01 area-%.

As used herein, “limit of detection (LOD)” refers to the lowest concentration of analyte that can be clearly detected above the base line signal, is estimated is three times the signal to noise ratio.

The term “micronization” used herein means a process or method by which the size of a population of particles is reduced.

As used herein, the term “micron” or “nm” both are equivalent refers to “micrometer” which is 1×10⁻⁶ meter.

As used herein, “crystalline particles” means any combination of single crystals, aggregates and agglomerates.

As used herein, “Particle Size Distribution (PSD)” means the cumulative volume size distribution of equivalent spherical diameters as determined by laser diffraction in Malvern Master Sizer 2000 equipment or its equivalent.

The important characteristics of the PSD are the (D₉₀), which is the size, in microns, below which 90% of the particles by volume are found, and the (D₅₀), which is the size, in microns, below which 50% of the particles by volume are found. Thus, a D₉₀ or d(0.9) of less than 300 microns means that 90 volume-percent of the particles in a composition have a diameter less than 300 microns.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. Imatinib or a pharmaceutically acceptable salt thereof comprising a N-(2-methyl-5-methylamino-phenyl)-N-(4-pyridin-3-yl-pyrimidin-2-yl)-formamide (formamide impurity) in an amount of less than about 1.5 area-% as measured by HPLC.

2. Imatinib of claim 1, having a purity of about 98.5 area-% to about 99.99 area-% as measured by HPLC.

3. Imatinib of claim 1, comprising the formamide impurity in an amount of about 0.01 area-% to about 1.5 area-%.

4. Imatinib of claim 1, further comprising a 4-[4-(imidazole-1-carbonyl)-piperazin-1-ylmethyl]-N-[4-methyl-3-(4-pyridin-3-yl-pyrimidin-2-ylamino)-phenyl]-benzamide (carbonylimidazole impurity) in an amount of about 0.01 area-% to about 0.15 area-% as measured by HPLC.

5. Imatinib of claim 1, wherein the pharmaceutically acceptable salt of imatinib is a hydrochloride, a hydrobromide, an oxalate, a maleate, a fumarate, a mesylate, a besylate, a tosylate or a tartrate salt.

6. Imatinib of claim 5, wherein the pharmaceutically acceptable salt of imatinib is imatinib mesylate.

7. An isolated formamide compound, N-(2-methyl-5-methylamino-phenyl)-N-(4-pyridin-3-yl-pyrimidin-2-yl)-formamide, of formula 1: 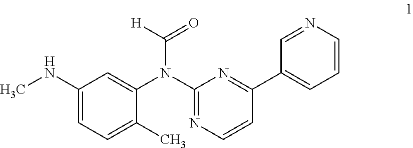
or an acid addition salt thereof.

8. An isolated carbonylimidazole compound, 4-[4-(imidazole-1-carbonyl)-piperazin-1-ylmethyl]-N-[4-methyl-3-(4-pyridin-3-yl-pyrimidin-2-ylamino)-phenyl]-benzamide, of formula 2: 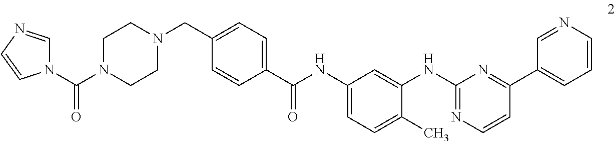
or an acid addition salt thereof.

9. A purification process for obtaining highly pure imatinib or a pharmaceutically acceptable salt thereof of any one of claim 1, comprising:
a) contacting crude imatinib base with a dihydrogen phosphate in a solvent medium comprising water and a first organic solvent to produce a biphasic reaction mixture, wherein the first organic solvent is selected from the group consisting of an alcohol, a ketone, a chlorinated hydrocarbon, a hydrocarbon, an ester, a nitrile, an ether, a polar aprotic solvent, and mixtures thereof
b) separating the aqueous layer from the biphasic reaction mixture and adding a second organic solvent to the separated aqueous layer to produce a reaction mixture, wherein the second organic solvent is selected from the group consisting of an alcohol, a ketone, a chlorinated hydrocarbon, a hydrocarbon, an ester, a nitrile, an ether, a polar aprotic solvent, and mixtures thereof;
c) combining the reaction mixture obtained in step-(b) with a base to produce a reaction mass; and
d) isolating and/or recovering the highly pure imatinib free base substantially free of impurities as a solid from the reaction mass;
e) optionally, suspending the highly pure imatinib free base obtained in step-(d) in a third organic solvent and recovering the imatinib free base from the suspension, wherein the third organic solvent is selected from the group consisting of an aliphatic or aromatic hydrocarbon, an ether, an alcohol, a ketone, and mixtures thereof;
f) optionally, converting the highly pure imatinib obtained in step-(d) or step-(e) into a pharmaceutically acceptable salt thereof.

10. The process of claim 9, wherein the first and second organic solvents are, each independently, selected from the group consisting of methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, methylene chloride, ethylene dichloride, chloroform, n-pentane, n-hexane, n-heptane, cyclohexane, toluene, xylene, tetrahydrofuran, dioxane, diethyl ether, diisopropyl ether, monoglyme, diglyme, N,N-dimethylformamide, N,N-dimethylacetamide, dimethylsulfoxide, and mixtures thereof; and wherein the third organic solvent is selected from the group consisting of n-pentane, n-hexane, n-heptane, cyclohexane, toluene, xylene, tetrahydrofuran, dioxane, diethyl ether, diisopropyl ether, monoglyme, diglyme, methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, and mixtures thereof.

11. The process of claim 10, wherein the first organic solvent is selected from the group consisting of methanol, ethanol, isopropyl alcohol, acetone, methylene chloride, N,N-dimethylformamide, N,N-dimethylacetamide, dimethylsulfoxide, and mixtures thereof; wherein the second organic solvent is an ester solvent selected from the group consisting of ethyl acetate, methyl acetate, isopropyl acetate, tert-butyl methyl acetate, ethyl formate, and mixtures thereof; and wherein the third organic solvent is a hydrocarbon solvent selected from the group consisting of n-pentane, n-hexane, n-heptane, cyclohexane, toluene, xylene, and mixtures thereof.

12. The process of claim 9, wherein the dihydrogen phosphate used in step-(a) is selected from the group consisting of sodium dihydrogen phosphate, potassium dihydrogen phosphate and ammonium dihydrogen phosphate; and wherein the base used in step-(c) is an inorganic base or an organic base selected from the group consisting of triethyl amine, dimethyl amine, tert-butyl amine, aqueous ammonia, sodium hydroxide, calcium hydroxide, magnesium hydroxide, potassium hydroxide, lithium hydroxide, sodium carbonate, potassium carbonate, lithium carbonate, sodium bicarbonate, potassium bicarbonate, sodium tert-butoxide, sodium isopropoxide and potassium tert-butoxide.

13. The process of claim 9, wherein the contacting in step-(a) is carried out under stirring at a temperature of below about reflux temperature of the solvent medium used for at least 10 minutes; wherein the pH of the reaction mass in step-(a) is adjusted between 4 and 5 during the addition of dihydrogen phosphate; wherein the crude imatinib base used in step-(a) is in the form of a solid, a solution, a suspension or a reaction mass containing crude imatinib base; wherein the combining in step-(c) is accomplished by adding the reaction mixture to the base or by adding the base to the reaction mixture; wherein the isolation of highly pure imatinib free base in step-(d) is carried out by cooling, seeding, partial removal of the solvent from the solution, by combining an anti-solvent with the solution, or a combination thereof; and wherein the recovery of highly pure imatinib free base in step-(d) is accomplished by filtration, filtration under vacuum, decantation, centrifugation, filtration employing a filtration media of a silica gel or celite, or a combination thereof.

14. The process of claim 13, wherein the crystallization is carried out by cooling the solution at a temperature of about 0° C. to about 20° C. for about 30 minutes to about 20 hours.

15. A process preparing crystalline form-α of imatinib mesylate, comprising:
a) providing a solution of imatinib mesylate in a solvent selected from the group consisting of N,N-dimethylformamide, N,N-dimethylacetamide and mixtures thereof;
b) optionally, filtering the solution to remove insoluble matter;
c) precipitating crystalline form-α of imatinib mesylate by combining the solution obtained in step-(a) or step-(b) with an anti-solvent selected from the group consisting of isopropyl alcohol, acetone and mixtures thereof; and
d) optionally, seeding the solution in step-(c) with crystalline form-α prior to or after the addition of anti-solvent;
e) recovering the crystalline form-α of imatinib mesylate in substantially pure form.

16. The process of claim 15, wherein the solution in step-(a) is provided either i) by dissolving imatinib mesylate in the solvent at a temperature of below about reflux temperature of the solvent; or ii) by admixing imatinib base, methanesulfonic acid and the solvent to obtain a mixture; and stirring the mixture to obtain a solution of imatinib mesylate; wherein the solution obtained in step-(a) is optionally subjected to carbon treatment or silica gel treatment; and wherein the recovery of imatinib mesylate crystalline form-α in step-(e) is accomplished by filtration, filtration under vacuum, decantation, centrifugation, filtration employing a filtration media of a silica gel or celite, or a combination thereof.

17. A pharmaceutical composition comprising imatinib or a pharmaceutically acceptable salt thereof comprising a N-(2-methyl-5-methylamino-phenyl)-N-(4-pyridin-3-yl-pyrimidin-2-yl)-formamide (formamide impurity) in an amount of less than about 1.5 area-% (as measured by HPLC), and one or more pharmaceutically acceptable excipients.

18. The pharmaceutical composition of claim 17, wherein the imatinib or a pharmaceutically acceptable salt thereof contains the formamide impurity in an amount of about 0.01 area-% to about 1.5 area-%, and further contains a 4-[4-(imidazole-1-carbonyl)-piperazin-1-ylmethyl]-N-[4-methyl-3-(4-pyridin-3-yl-pyrimidin-2-ylamino)-phenyl]-benzamide (carbonylimidazole impurity) in an amount of less than about 0.15 area-%.

19. The pharmaceutical composition of claim 18, wherein the imatinib or a pharmaceutically acceptable salt thereof contains the carbonylimidazole impurity in an amount of about 0.01 area-% to about 0.15 area-%.

20. The pharmaceutical composition of claim 17, wherein the imatinib or a pharmaceutically acceptable salt thereof has a D90 particle size of less than or equal to about 500 microns.

21. The pharmaceutical composition of claim 20, wherein the D₉₀ particle size is about 1 micron to about 300 microns, or about 10 microns to about 150 microns.

22. A method for treating a patient suffering from tumoral diseases, comprising administering a therapeutically effective amount of the highly pure imatinib or a pharmaceutically acceptable salt thereof of claim 1.
